(19) United States
(12) Patent Application Publication
(10) Pub. No.: US 2009/0149660 A1
(43) Pub. Date: Jun. 11, 2009

(54) OPTICAL RECORDING MATERIAL AND OPTICAL RECORDING MEDIA

(75) Inventors: Masahiro Shinkai, Tokyo (JP); Atsushi Monden, Tokyo (JP); Motohiro Inoue, Tokyo (JP)

Correspondence Address: OLIFF & BERRIDGE, PLC, P.O. BOX 320850, ALEXANDRIA, VA, 22320-4850, US

(73) Assignee: TDK CORPORATION, Tokyo (JP)

(21) Appl. No.: 12/067,754

(22) PCT Filed: Sep. 25, 2006
(86) PCT No.: PCT/JP2006/318938

§ 371 Date: Mar. 21, 2008

(30) Foreign Application Priority Data
Sep. 27, 2005 (JP) 2005-280735

(51) Int. Cl. C07D 209/56

(52) U.S. Cl. 548/427

(57) ABSTRACT

An optical recording medium provided with a recording layer that comprises a cation represented by the following general formula (1) and a chelate compound of an azo compound represented by the following general formula (2) and a metal.

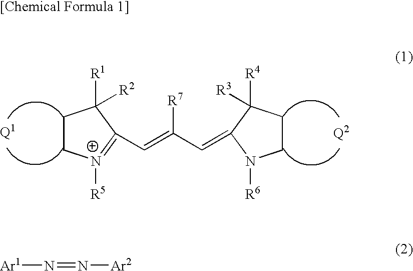

In the formulas, R¹-R⁴ each independently represent a monovalent group represented by Chemical Formula (10) below or other groups, R⁵ and R⁶ each independently represent an optionally substituted alkyl group or other groups, R⁷ represents a hydrogen atom or other groups, Q¹ and Q² each independently represent a group that forms an optionally substituted benzene ring or other groups, at least one from among R¹-R⁴ is a monovalent group represented by Chemical Formula (10) below,

[Chemical Formula 2]

[CH₂═CH—CH₂ (10)

and at least one of Ar¹ and Ar² is an aryl or other group having a substituent capable of coordinating with a metal atom.

### TECHNICAL FIELD

The present invention relates to an optical recording medium for recording of information by light exposure, and to an optical recording material employed in the same.

### BACKGROUND ART

Optical recording disks such as CD-R (write-once read-many type CD) and DVD-R (write-once read-many type DVD) disks are widely popular as optical recording media, and the wavelengths of the recording and reproducing beam are becoming increasingly smaller in order to achieve even higher recording densities. For example, the current recording and reproduction wavelength for CD-R disks is 780 nm r, but the next generation CD-R or DVD-R disks use shorter wavelengths of 635 to 680 nm. The pigments used in optical recording media that are known to respond to such short wavelength light include cyanine pigments (for example, see Patent document 1).

[Patent document 1] Japanese Unexamined Patent Publication HEI No. 11-34499

### DISCLOSURE OF THE INVENTION

### Problems to be Solved by the Invention

Optical recording media must also be suitable for high-speed recording, in addition to short wavelengths as mentioned above. More highly sensitive pigments are desirable for greater speeds, but higher pigment sensitivity also tends to increase jitter in the time direction of the reproduction signal, and lower preservation stability.

The present invention, which has been accomplished in light of the current circumstances, has as its object to provide an optical recording medium that has satisfactory sensitivity while exhibiting adequate characteristics in terms of jitter and preservation stability, as well as an optical recording material employed in the same.

### Means for Solving the Problems

The invention provides an optical recording material for use in an optical recording medium that allows recording of information by light exposure, the material comprising a cation represented by the following general formula (1) and a chelate compound of an azo compound represented by the following general formula (2) and a metal.

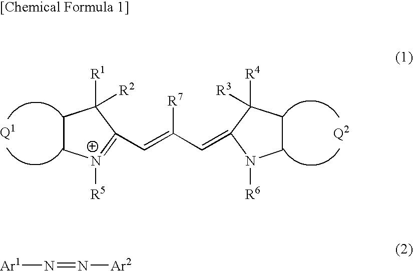

In formula (1), R¹ and R² each independently represent a monovalent group represented by Chemical Formula (10) below, a C1-4 alkyl group, an optionally substituted benzyl group, or a group linking together to form a 3- to 6-membered ring, R³ and R⁴ each independently represent a monovalent group represented by Chemical Formula (10) below, a C1-4 alkyl group, or a group linking together to form a 3- to 6-membered ring, R⁵ and R⁶ each independently represent an optionally substituted alkyl group or an optionally substituted aryl group, R⁷ represents a hydrogen atom, a halogen atom, a cyano group, an optionally substituted alkyl group or an optionally substituted aryl group, Q¹ and Q² each independently represent groups that form an optionally substituted benzene ring or an optionally substituted naphthalene ring, and at least one from among R¹, R², R³ and R⁴ is a monovalent group represented by Chemical Formula (10).

[Chemical Formula 2]

[CH₂═CH—CH₂ (10)

In formula (2), Ar¹ and Ar² each independently represent an optionally substituted aryl group, and at least one of Ar¹ and Ar² is an aryl group having a substituent capable of coordinating with a metal atom, or an aryl group composed of an optionally substituted nitrogen-containing heteroaromatic ring with a nitrogen atom capable of coordinating with a metal atom.

The optical recording material of the invention or an optical recording layer comprising an optical recording medium of the invention employs as the pigment a cation having the specific substituents mentioned above, and as a result of combining the aforementioned chelate compounds therewith in the specific proportions mentioned above, satisfactory sensitivity is achieved while sufficient characteristics are exhibited from the standpoint of jitter and preservation stability.

The optical recording material of the invention also preferably is obtainable by mixing a salt containing the aforementioned cation or its counter anion with the aforementioned chelate compound, and the optical recording layer comprising the optical recording medium of the invention preferably contains a mixture obtainable by mixing a salt of the aforementioned cation and its counter anion with the aforementioned chelate compound.

The optical recording material and optical recording medium can be more efficiently produced since they are obtainable by simply mixing the two different materials. The optical recording material and optical recording medium obtained by such a mixture may contain the counter anion of the aforementioned cation and the counter cation of the chelate compound, and such counter anions and counter cations have in the prior art tended to act as impurities that impair the quality stability of the optical recording material. However, the present inventors have discovered that such problems are rare in the optical recording material and optical recording medium of the invention that comprise the aforementioned specific pigment.

### EFFECT OF THE INVENTION

According to the invention there is provided an optical recording medium that has satisfactory sensitivity while exhibiting adequate characteristics in terms of jitter and shelf life, as well as an optical recording material employed in the same.

### BRIEF DESCRIPTION OF THE DRAWINGS

FIG. 1 is a cross-sectional view of an embodiment of an optical recording disk employing an optical recording medium according to the invention.

### EXPLANATION OF SYMBOLS

10: Base, 20: first recording layer, 30: semi-transparent reflective layer, 40: spacer layer, 50: second recording layer, 60: reflective layer, 70: adhesive layer, 80: dummy base, 12, 42: groove, 100: optical recording medium.

### BEST MODES FOR CARRYING OUT THE INVENTION

Preferred embodiments of the invention will now be explained in detail, with reference to the accompanying drawings as necessary. However, the present invention is not limited to the embodiments described below.

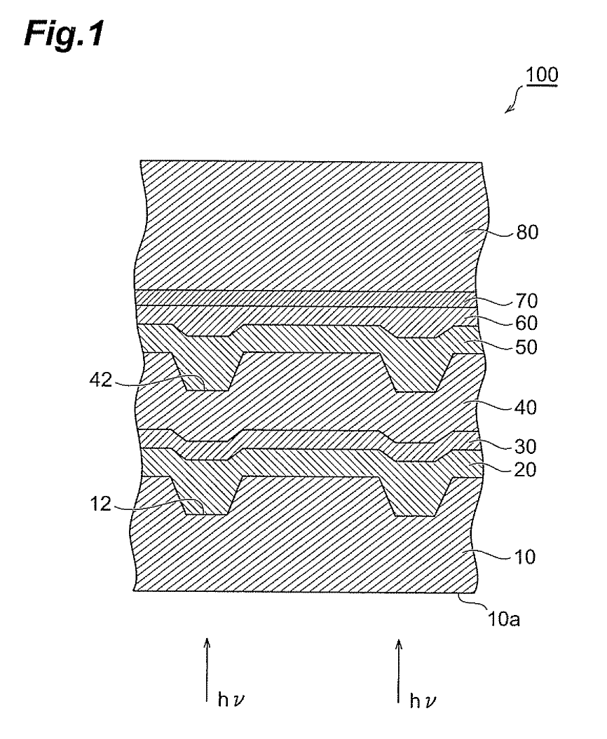

FIG. 1 is a cross-sectional view of an embodiment of an optical recording medium according to the invention. The optical recording medium 100 shown in FIG. 1 has a laminated structure comprising a first recording layer 20, semi-transparent reflective layer 30, spacer layer 40, second recording layer 50, reflective layer 60, adhesive layer 70 and dummy base 80 laminated in that order on a base 10. The optical recording medium 100 is a write-once read-many type optical recording disk capable of recording and reproduction of information using light with short wavelengths of 630 to 685 nm. The recording and reproducing beams are irradiated onto the optical recording medium 100 from the base 10 side (the lower side in the drawing).

During recording of information, the optical recording medium 100 is irradiated with the recording beam in a pulse fashion from the outer surface 10a of the base 10 side. Appropriate focusing during this time causes selective absorption of light energy at the prescribed sections of the first recording layer 20 or second recording layer 50, thus altering the optical reflectance at those sections. Recording of information is accomplished by this alteration in optical reflectance.

The base 10 and dummy base 80 are disk-shaped, with a diameter of about 64 to 200 mm and a thickness of about 0.6 mm each. The base 10 is preferably one that is substantially transparent to the recording and reproducing beams, and more specifically, the transmittance of the base 10 for the recording and reproducing beams is preferably at least 88%. The materials of the base 10 and dummy base 80 are preferably resins or glass, among which thermoplastic resins such as polycarbonate resins, acrylic resins, amorphous polyethylene, TPX, polystyrene-based resins and the like are particularly preferred. The dummy base 80 does not necessarily need to be transparent.

A tracking groove 12 is formed on the first recording layer 20 side of the base 10. The groove 12 is preferably a spiral continuous groove, preferably with a depth of 0.1 to 0.25 [symbol] m, a width of 0.20 to 0.50 [symbol] m and a groove pitch of 0.6 to 1.0 [symbol] m. A groove with such a structure will allow a satisfactory tracking signal to be obtained without lowering the reflection level of the groove. The groove 12 may be formed simultaneously with formation of the base 10, by injection molding or the like using the aforementioned resin. Alternatively, a resin layer with a groove may be formed by the “2P” method in which the groove shape is transferred to a flat base from a resin stamper or the like having a raised section corresponding to the groove shape, to obtain the base 10 as a composite base comprising the base and the resin layer.

At least one of the first recording layer 20 and second recording layer 50 is composed of an optical recording material comprising a cation represented by general formula (1) above (hereinafter also referred to as “trimethinecyanine pigment cation”) and a chelate compound of an azo compound and a metal. The compositions of the optical recording materials composing the first recording layer 20 and second recording layer 50 may be the same or different.

In formula (1), R¹ and R² each independently represent a monovalent group represented by Chemical Formula (10) above, a C1-4 alkyl group, an optionally substituted benzyl group, or a group linking together to form a 3- to 6-membered ring, and R³ and R⁴ each independently represent a monovalent group represented by Chemical Formula (10), a C1-4 alkyl group, an optionally substituted benzyl group, or a group linking together to form a 3- to 6-membered ring. The trimethinecyanine pigment cation is in a state of equilibrium between the structure of formula (1) and the structure of the following formula (1′).

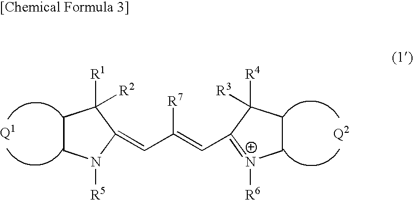

At least one of R¹-R⁴ is a monovalent group represented by Chemical Formula (10) (hereinafter referred to as “allyl group”). Preferably, R¹ is an allyl group, R², R³ and R⁴ are C1-4 alkyl or optionally substituted benzyl groups, R¹ and R² are allyl groups and R³ and R⁴ are C1-4 allyl or optionally substituted benzyl groups, R¹ and R³ are allyl groups and R² and R⁴ are C1-4 alkyl or optionally substituted benzyl groups, or R¹, R² and R³ are allyl groups and R⁴ is a C1-4 alkyl or optionally substituted benzyl group. Most preferably, R¹ is an allyl group and R², R³ and R⁴ are C1-4 alkyl or optionally substituted benzyl groups, or R¹ and R³ are allyl groups and R² and R⁴ are C1-4 alkyl or optionally substituted benzyl groups.

When R¹-R⁴ are C1-4 alkyl groups, they are preferably methyl, ethyl or n-propyl groups. When R¹-R⁴ are optionally substituted benzyl groups, they are preferably benzyl groups with the benzene rings substituted with a methyl group or a halogen atom, or unsubstituted benzyl groups. When R¹ and R² or R³ and R⁴ link together to form 3- to 6-membered rings, they preferably form cyclopropane rings, cyclobutane rings, cyclopentane rings or cyclohexane rings. At least one non-allyl group among R¹-R⁴ is preferably an optionally substituted benzyl group. This will help to further improve jitter.

R⁵ and R⁶ each independently represent an optionally substituted alkyl or optionally substituted aryl group. When R⁵ and R⁶ are optionally substituted alkyl groups, at least one of R⁵ and R⁶ is preferably a C1-5 alkyl group, from the viewpoint of improving solubility in the solvent used to form the recording layer. As specific preferred examples for R⁵ and R⁶ include a methyl, ethyl, n-pentyl, isopentyl, neopentyl, n-hexyl, isohexyl, n-heptyl, 5-methylhexyl, n-octyl, 3,4-dimethylpentyl and phenyl group. Among these, R⁵ and R⁶ preferably each independently represent a methyl, ethyl, n-propyl, isopropyl or isopentyl group.

R⁷ represents a hydrogen atom, a halogen atom, or a cyano group, an optionally substituted alkyl group or an optionally substituted aryl group. R⁷ is more preferably a hydrogen atom, a halogen atom, a C1-4 alkyl group, a cyano group, an optionally substituted phenyl group or an optionally substituted benzyl group, with a hydrogen atom being particularly preferred.

Q¹ and Q² each independently represent a group that forms an optionally substituted aromatic ring. The aromatic ring is fused with the ring to which Q¹ or Q² is bonded. Q¹ and Q² preferably form an optionally substituted benzene ring or optionally substituted naphthalene ring. Preferred substituents on the aromatic ring of Q¹ and Q² are methyl, ethyl, isopropyl, fluoro, chloro, bromo, methoxy, nitro and cyano groups.

More specifically, the trimethinecyanine pigment cation is preferably one represented by the following general formula (11), (12), (13), (14), (15), (16) or (17).

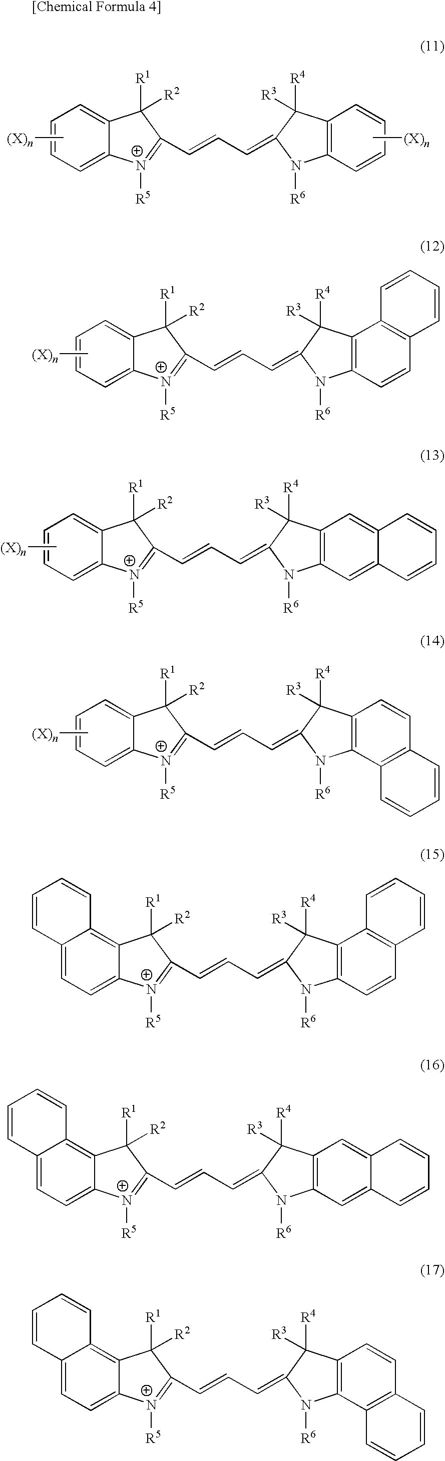

In formulas (11) to (17), R¹, R², R³, R⁴, R⁵ and R⁶ and their preferred examples are the same as R¹, R², R³, R⁴, R⁵ and R⁶ in formula (1). X represents a halogen atom, a nitro group, a hydroxyl group, an optionally substituted alkoxy group (preferably methoxy), an optionally substituted aryl group (preferably phenyl) or an optionally substituted alkyl group (preferably methyl, ethyl or trifluoromethyl), and multiple X groups in the same molecule may be the same or different. The letter n represents an integer of 1 to 4 (preferably 1 or 2).

As specific preferred examples of trimethinecyanine pigment cations represented by general formulas (11) to (17) there may be mentioned those represented by the following chemical formulas (T1) to (T58). They may be used alone or in combinations of two or more. These trimethinecyanine pigment cations can be synthesized by known processes using compounds with specified substituents as starting materials.

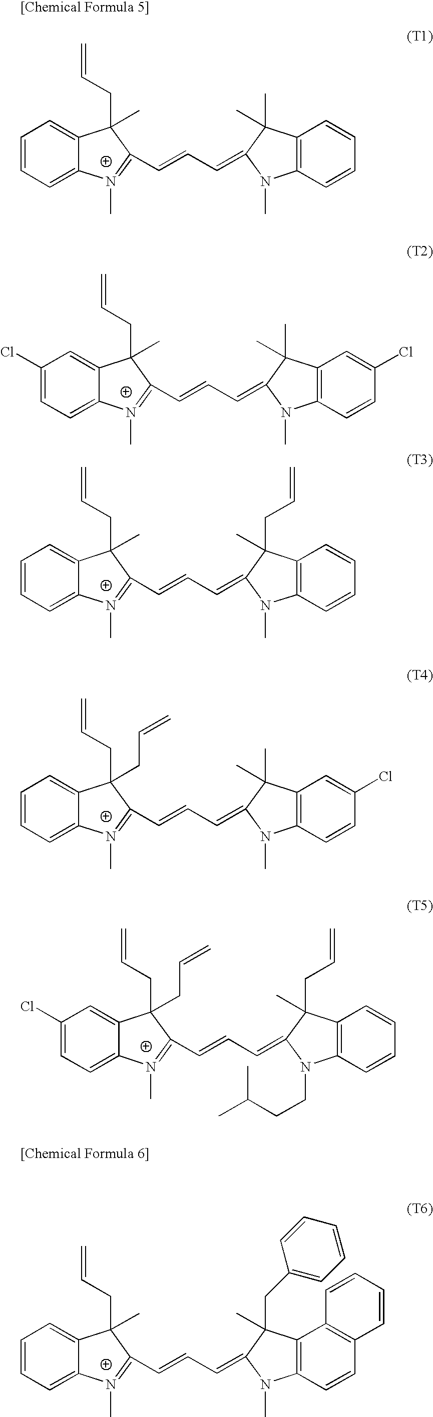

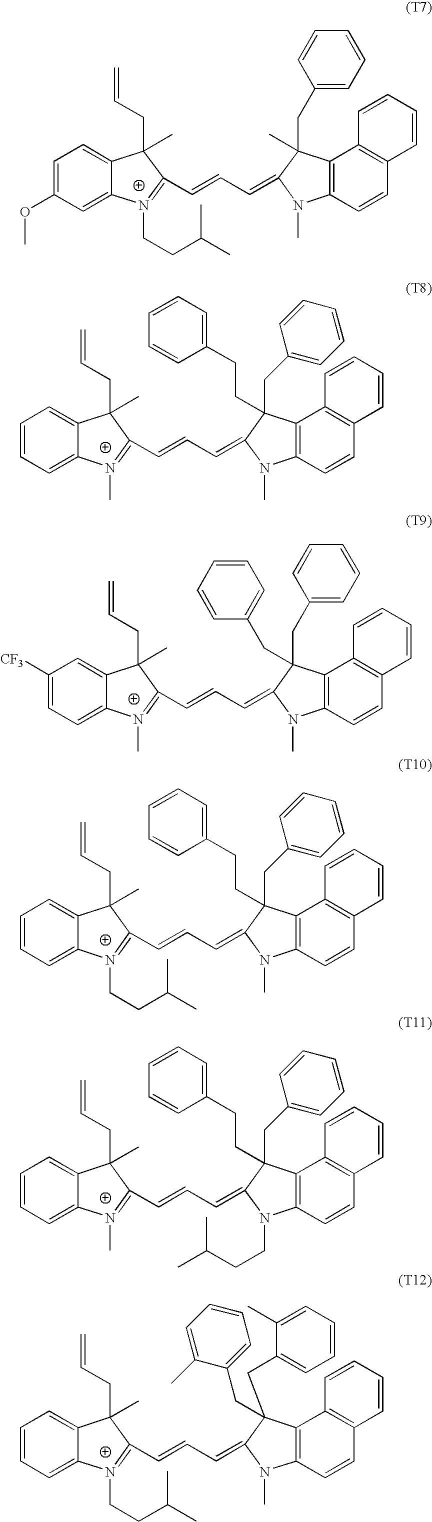

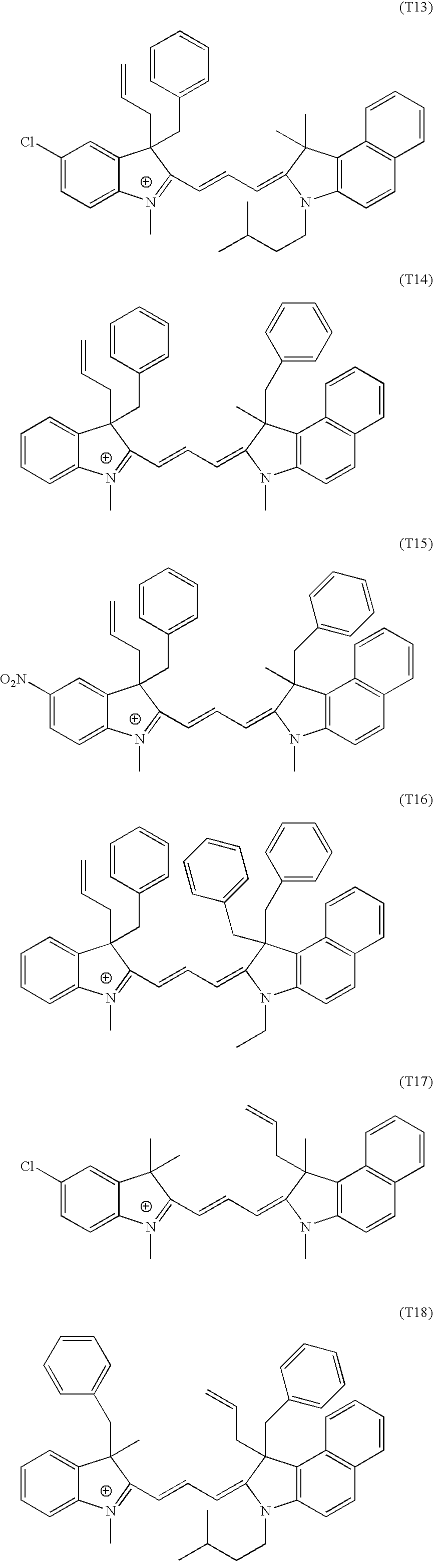

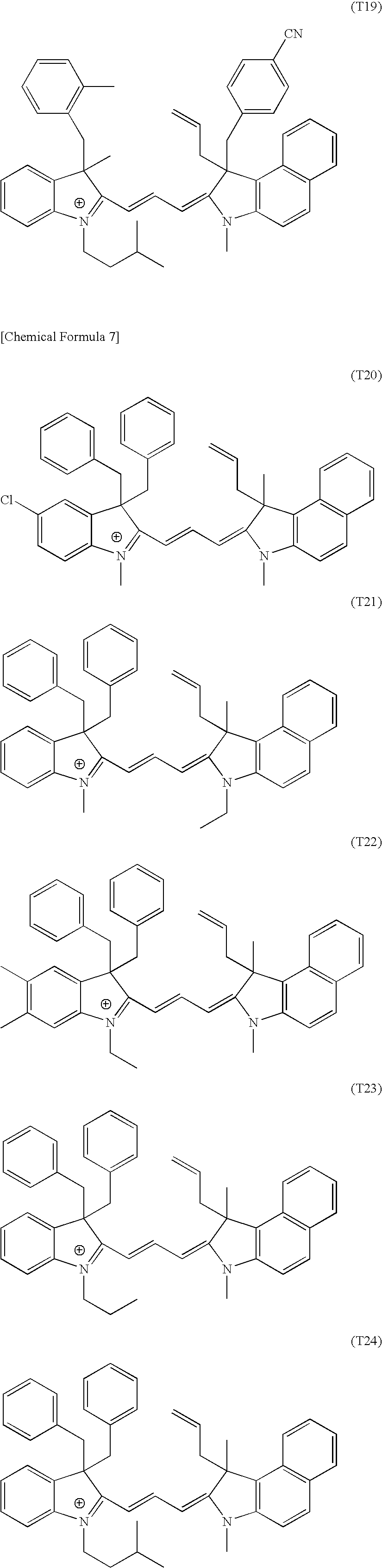

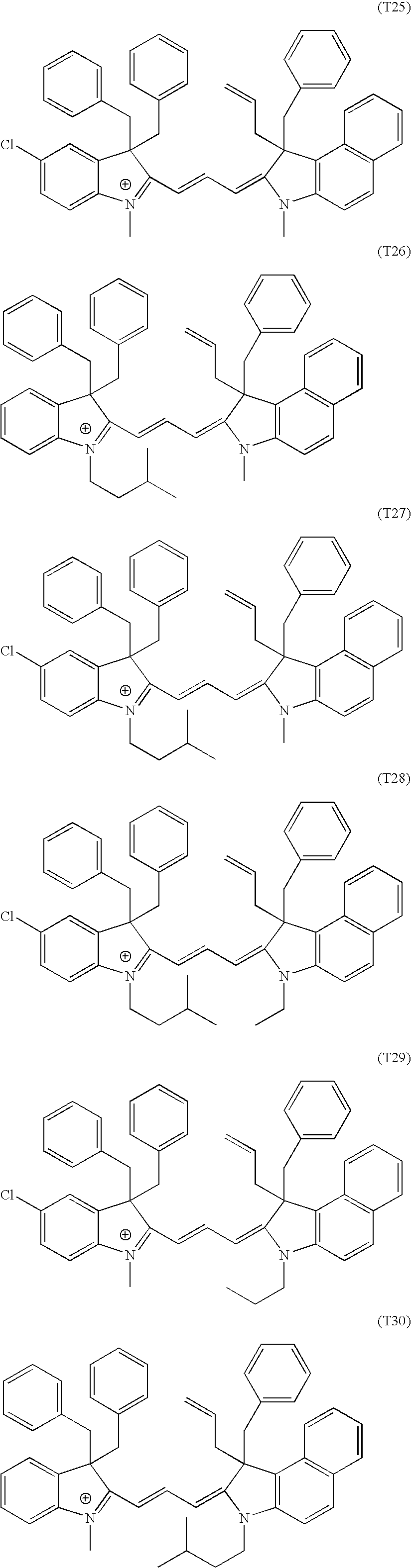

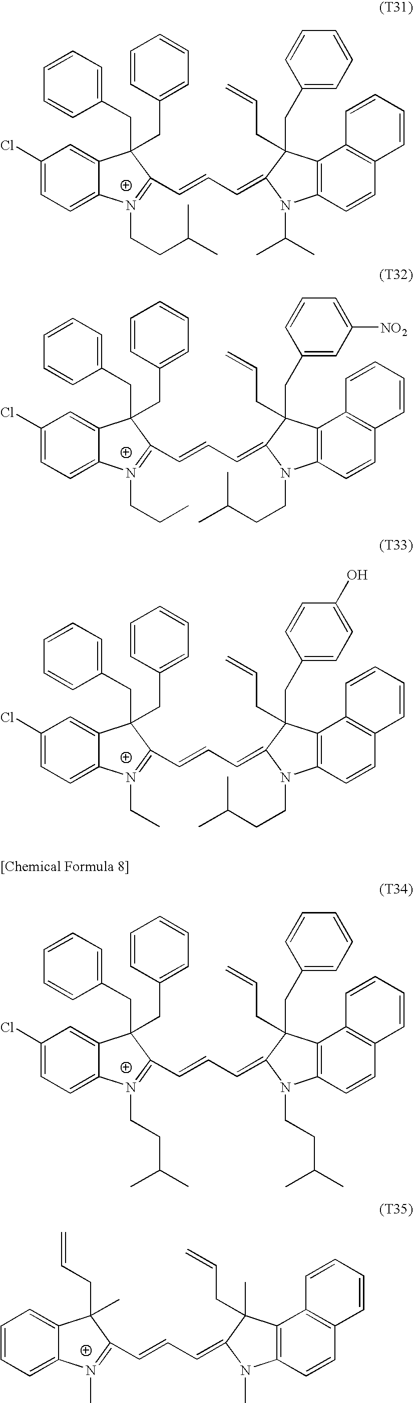

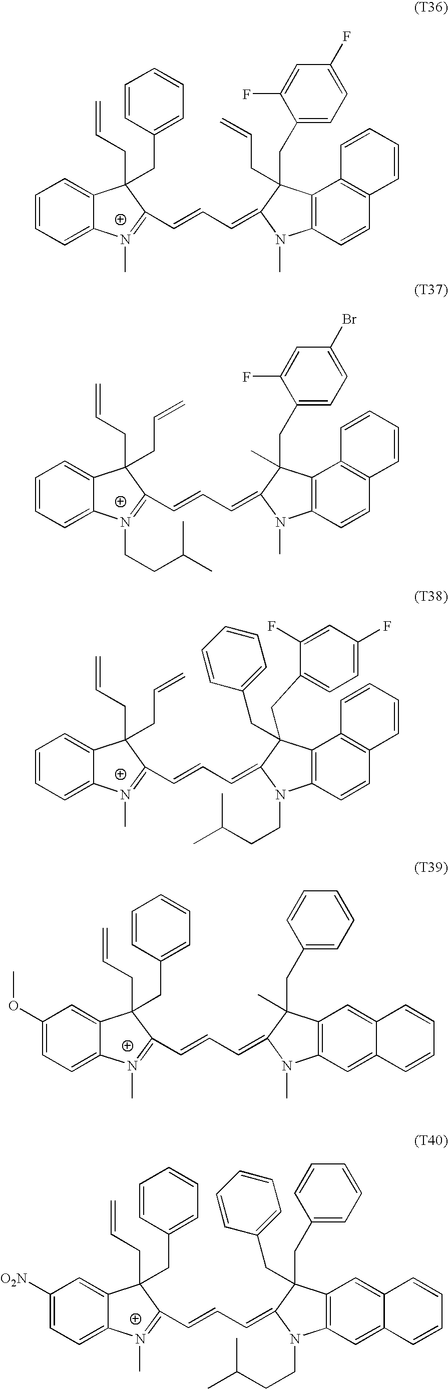

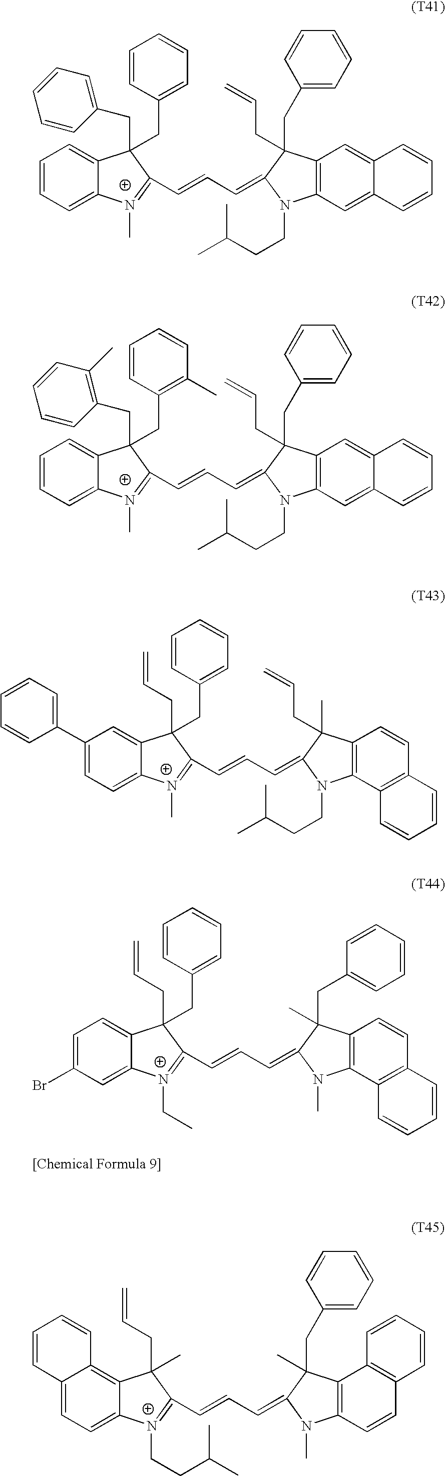

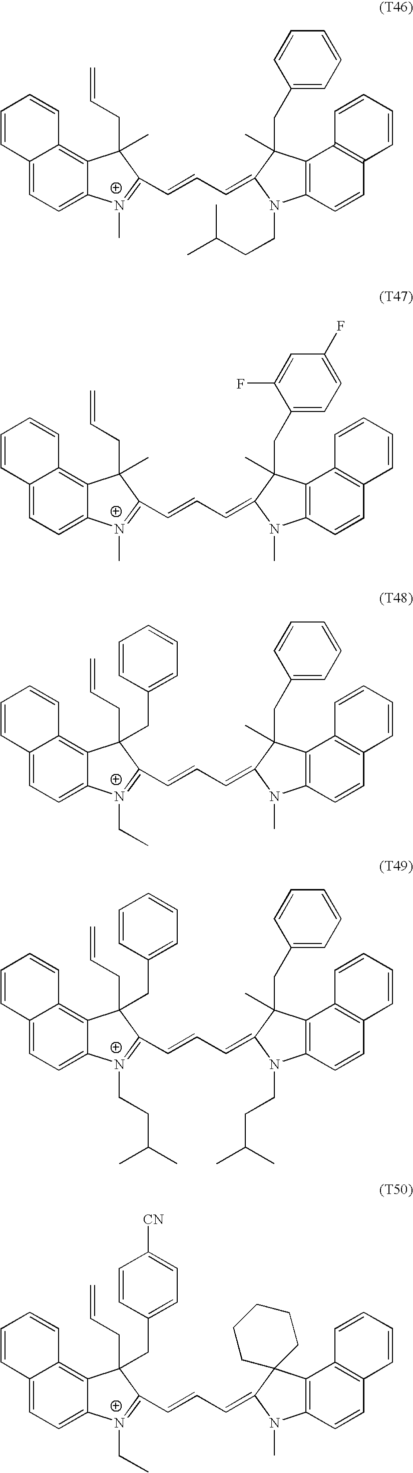

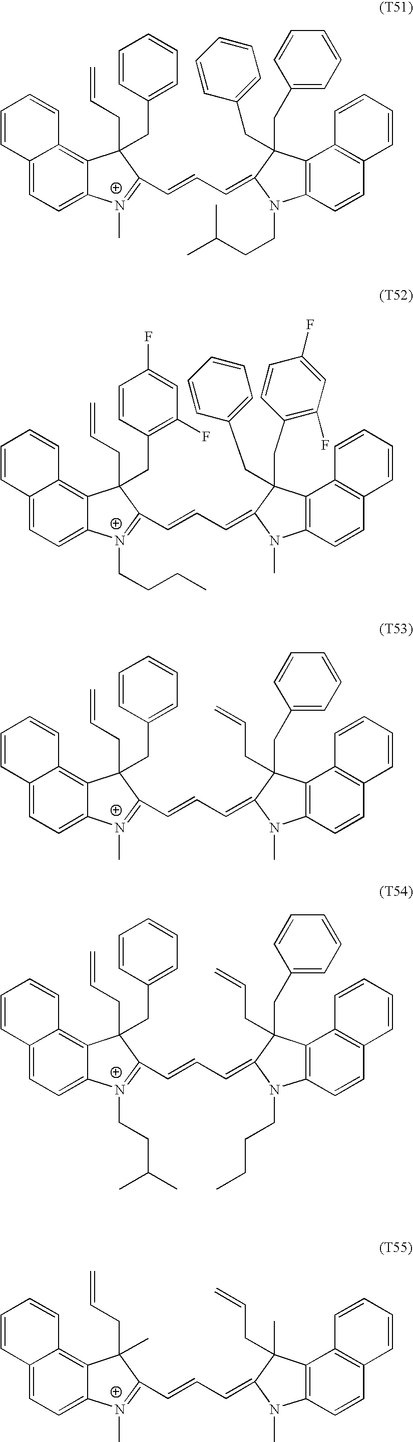

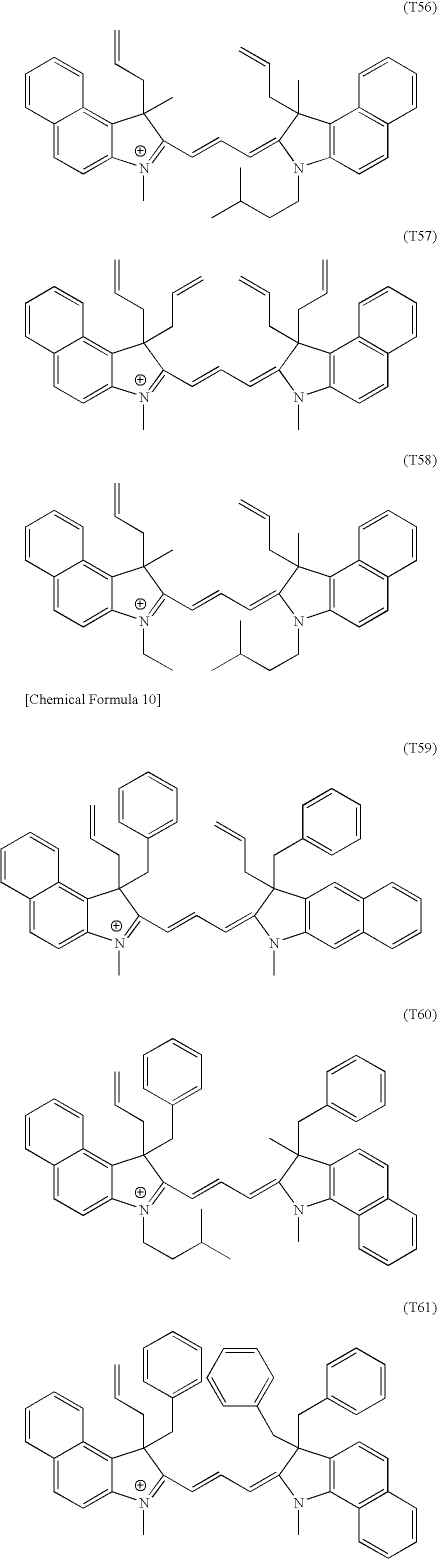

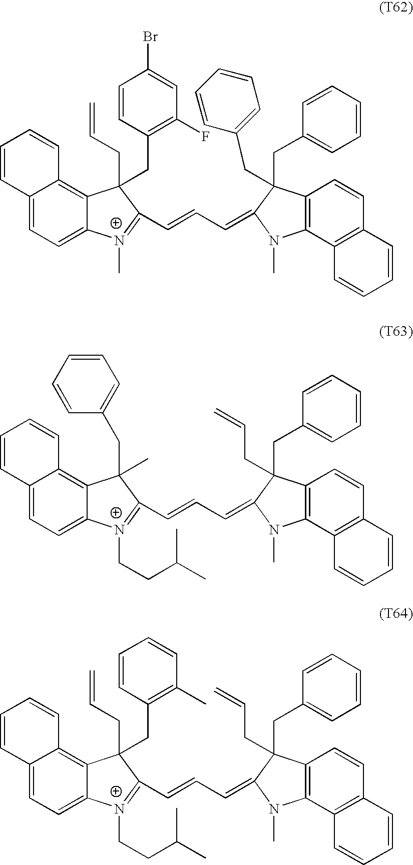

The trimethinecyanine pigment cations will usually be used in combination with counter anions that neutralize their positive charge. As examples of counter anions there may be mentioned monovalent anions such as ClO₄⁻, I⁻, BF₄⁻, PF₆⁻ and SbF₆⁻. When the chelate compound is an anion, it may be used as the counter anion of the trimethinecyanine pigment cation to form a salt. At least one of the anions PF₆⁻ and SbF₆⁻ is preferred from the standpoint of optimizing the leveling property.

The chelate compound is a metal chelate compound formed with the azo compound represented by formula (2) coordinated with a metal, and these are also known as azo-based pigments or azo-based dyes.

In formula (2), Ar¹ and Ar² each independently represent an optionally substituted aryl group, and at least one of them is an aryl group with a substituent capable of coordinating with a metal atom or an aryl group composed of an optionally substituted nitrogen-containing heteroaromatic ring with a nitrogen atom capable of coordinating with a metal atom. The substituent capable of coordinating with a metal atom and the nitrogen atom capable of coordinating with a metal atom are preferably at a position allowing coordination with the metal together with the azo group (for example, the ortho position in the case of a benzene ring).

Ar¹ and Ar² are monocyclic or fused polycyclic or linked polycyclic aromatic rings. As such aromatic rings there may be mentioned benzene, naphthalene, pyridine, thiazole, benzothiazole, oxazole, benzoxazole, quinoline, imidazole, pyrazine and pyrrole rings, among which benzene, pyridine, quinoline and thiazole rings are particularly preferred.

As substituents capable of coordinating with metal atoms there may be mentioned groups with active hydrogens. As groups with active hydrogens there may be mentioned hydroxyl, mercapto, amino, carboxyl, carbamoyl, optionally substituted sulfamoyl, sulfo and sulfonylamino, among which hydroxyl, primary or secondary amino groups and optionally substituted sulfamoyl groups are especially preferred. Ar¹ and Ar² may have a substituent in addition to the substituent capable of coordinating with a metal atom.

The substituents of Ar¹ and Ar² may be the same or different, and when they are different, Ar¹ preferably has at least one group selected from the group consisting of a nitro group, a halogen atom (for example, chlorine and bromine), a carboxyl group, a sulfo group, a sulfamoyl group and an alkyl groups (preferably C1-4 and more preferably methyl), and Ar² preferably has at least one group selected from the group consisting of an amino group (preferably dialkylamino groups with a total of 2-8 carbon atoms, examples of which include dimethylamino, diethylamino, methylethylamino, methylpropylamino, dibutylamino and hydroxyethylmethylamino), an alkoxy group (preferably C1-4, such as methoxy), an alkyl group (preferably C1-4 and more preferably methyl), an aryl group (preferably monocyclic, such as phenyl or chlorophenyl), a carboxyl group and a sulfo group. When Ar¹ is an optionally substituted phenyl group, the substituent is preferably at the meta or para position with respect to the azo group, and more preferably at the meta position.

More specifically, Ar¹ and Ar² are preferably monovalent groups represented by the following general formula (20a), (20b), (20c), (20d), (20e), (20f), (20g), (20 h) or (20i).

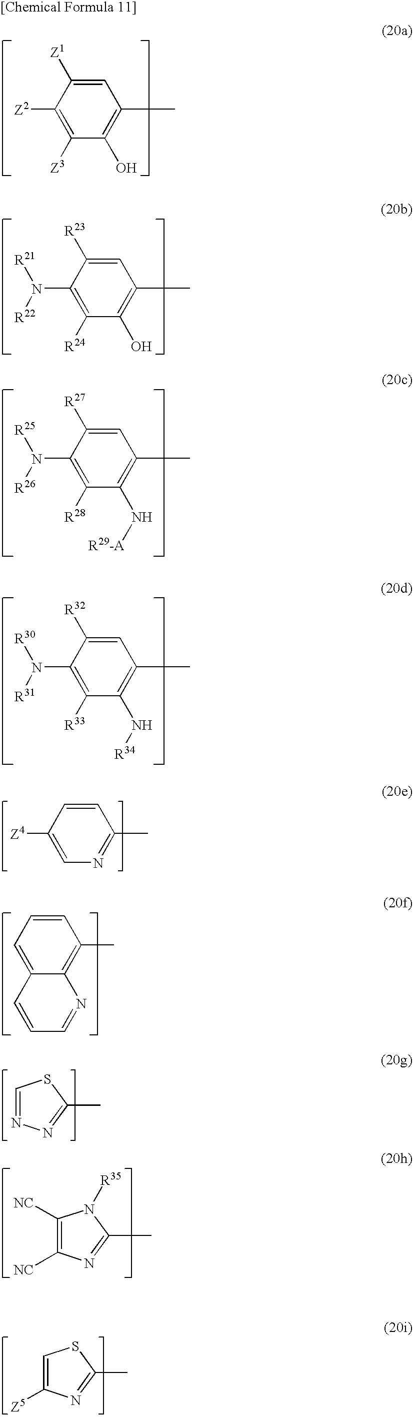

In formula (20a), Z¹, Z² and Z³ each independently represent a hydrogen atom, a halogen atom or a nitro group, and at least one of them is preferably a halogen atom or nitro group.

In formula (20b), R²¹, R²², R²³ and R²⁴ each independently represent an optionally substituted C2-8 alkyl or optionally substituted aryl group. R²¹ and R²³, and R²² and R²⁴ may be respectively linked to form a ring.

In formula (20c), R²⁵, R²⁶, R²⁷ and R²⁸ have the same preferred examples as R²¹, R²², R²³ and R²⁴ in formula (20b). R²⁹ represents an optionally substituted alkyl or optionally substituted aryl group. R²⁹ is preferably a C1-4 alkyl, trifluoromethyl, pentafluoroethyl, 2,2,2-trifluoroethyl, optionally substituted phenyl or optionally substituted benzyl group. The letter A represents a divalent group represented by —SO₂— or —CO—, and it is preferably the divalent group represented by —SO₂—.

In formula (20d), R³⁰, R³¹, R³² and R³³ have the same preferred examples as R²¹, R²², R²³ and R²⁴ in formula (20b). R³⁴ represents an optionally substituted alkyl or optionally substituted aryl group, and is preferably an optionally substituted C1-4 alkyl or optionally substituted phenyl group.

In formulas (20e) and (20i), Z⁴ and Z⁵ represent a hydrogen atom, a halogen atom or a nitro group, and preferably a halogen atom or a nitro group.

As preferred examples of azo compounds there may be mentioned those represented by the following chemical formulas (A1) to (A63).

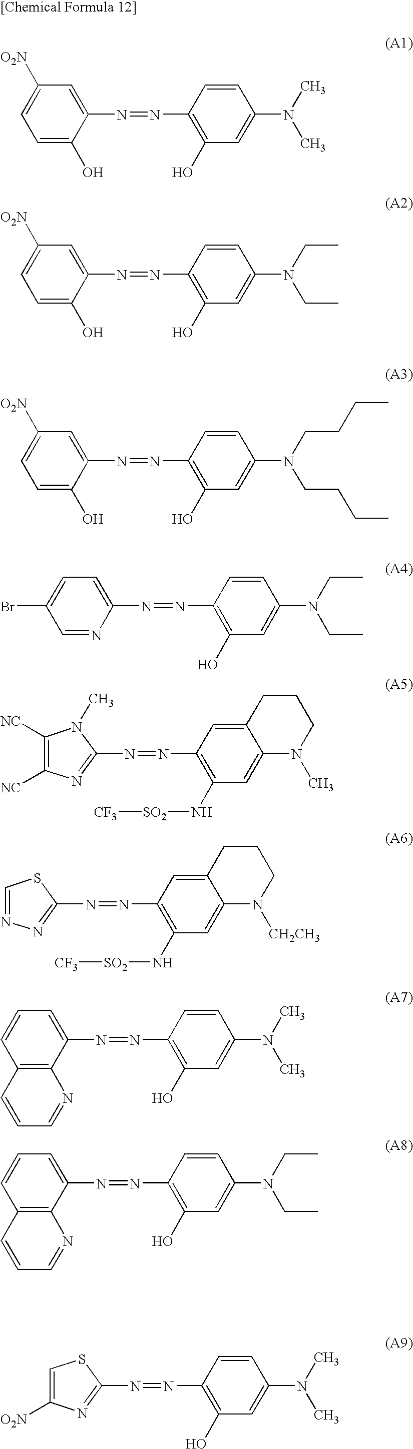

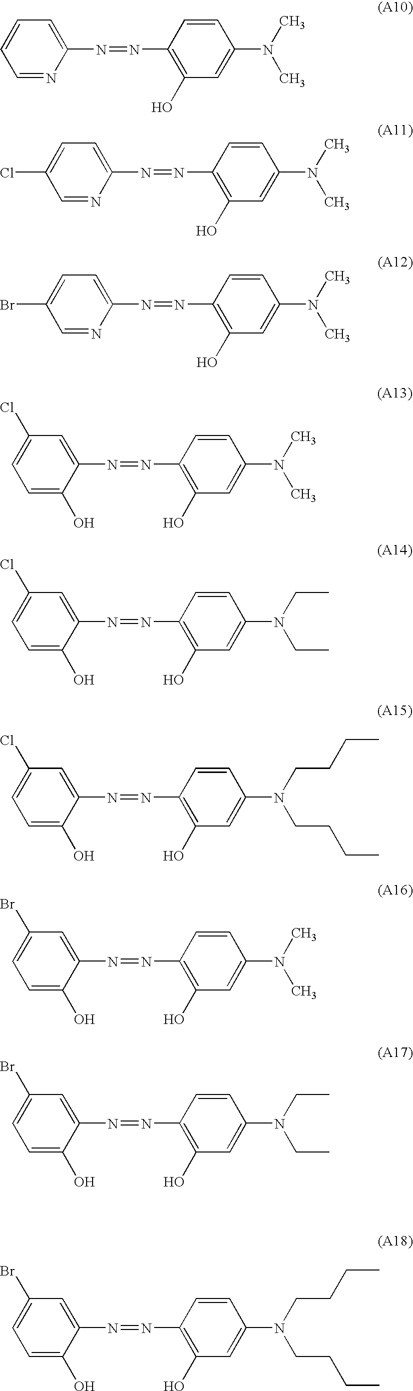

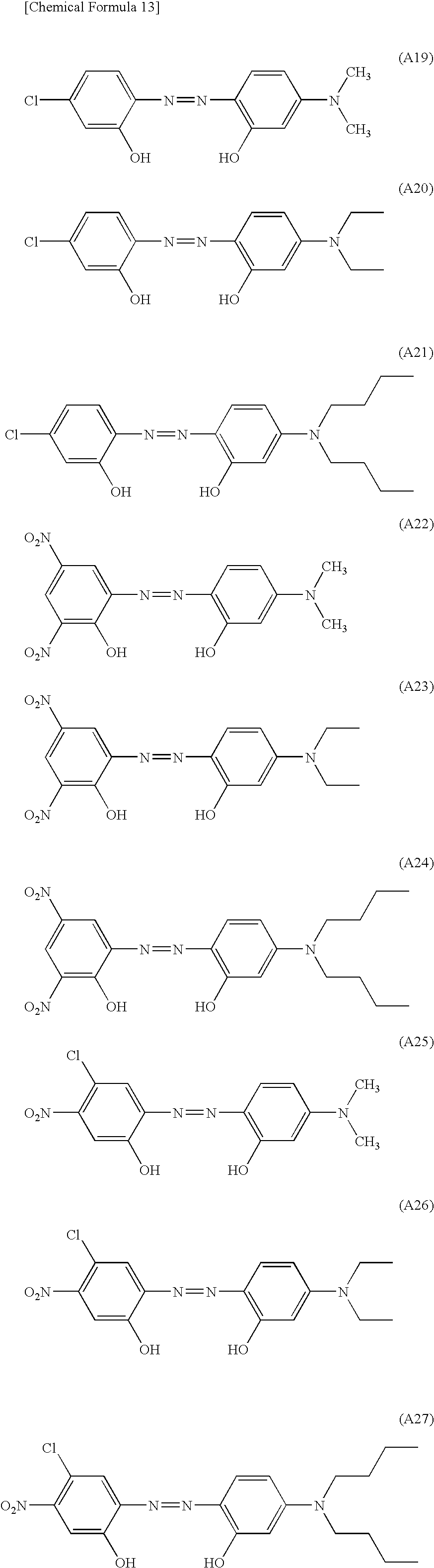

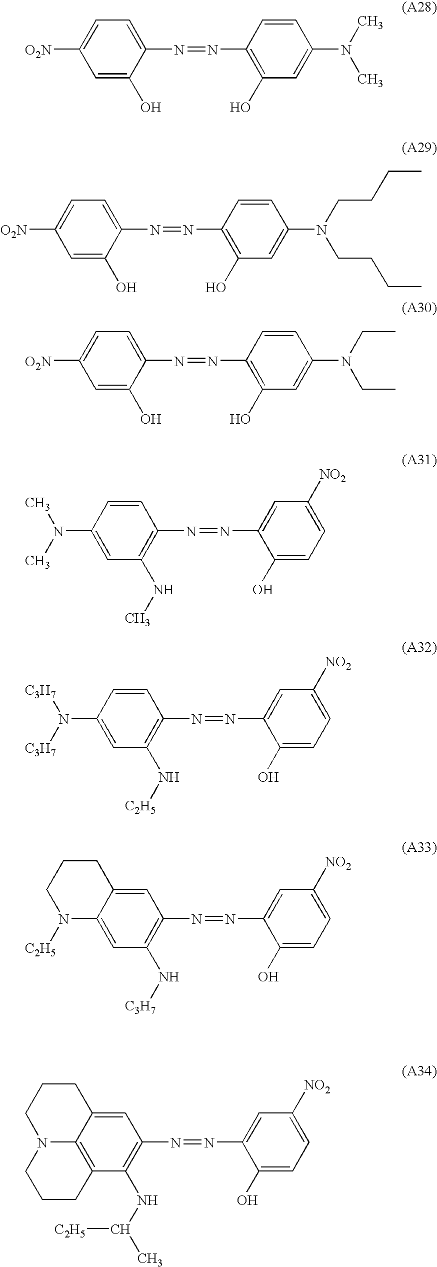

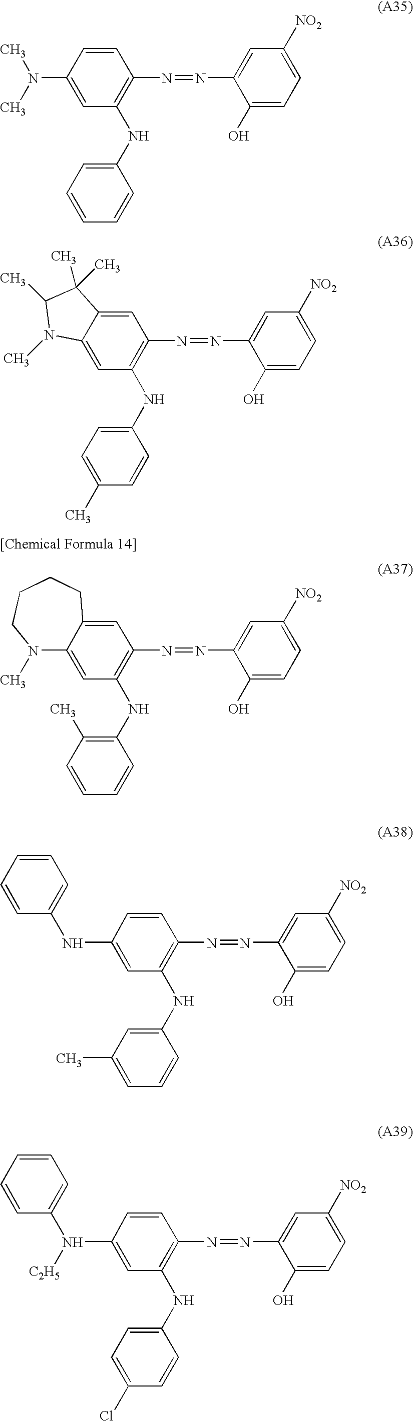

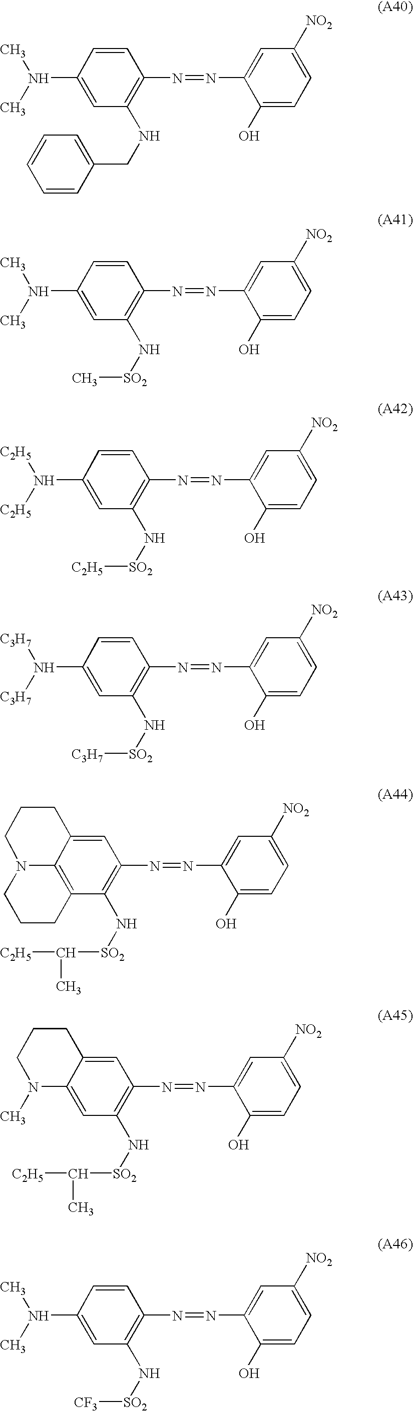

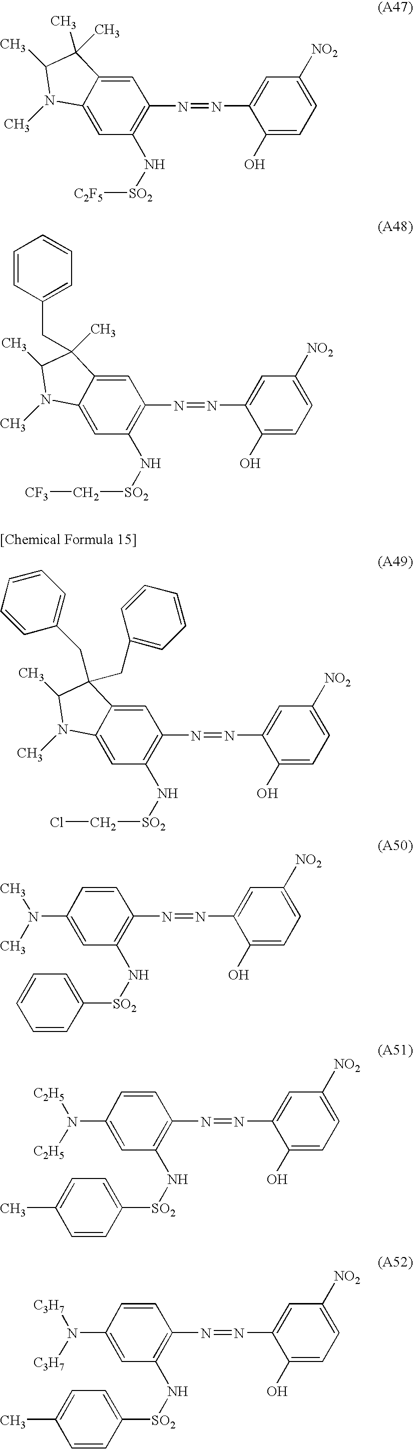

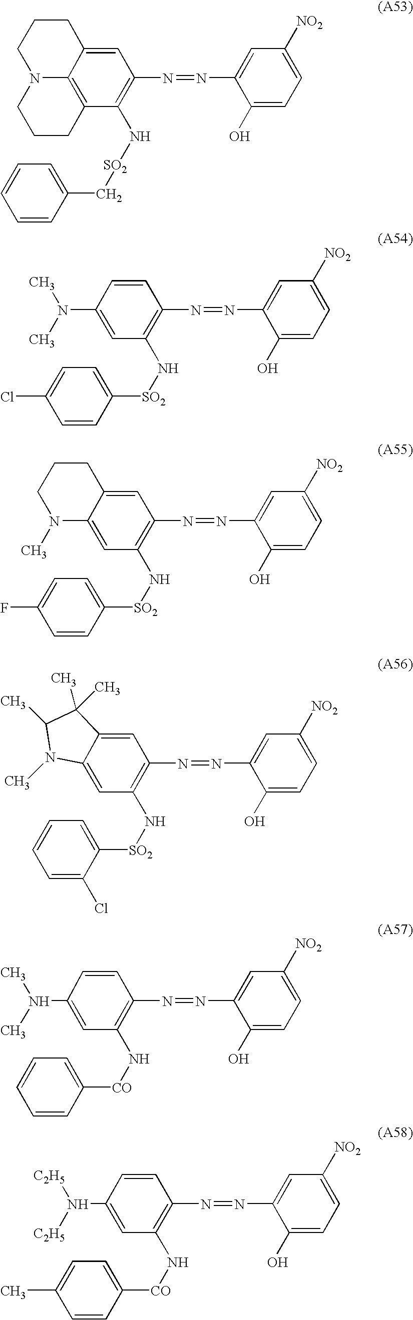

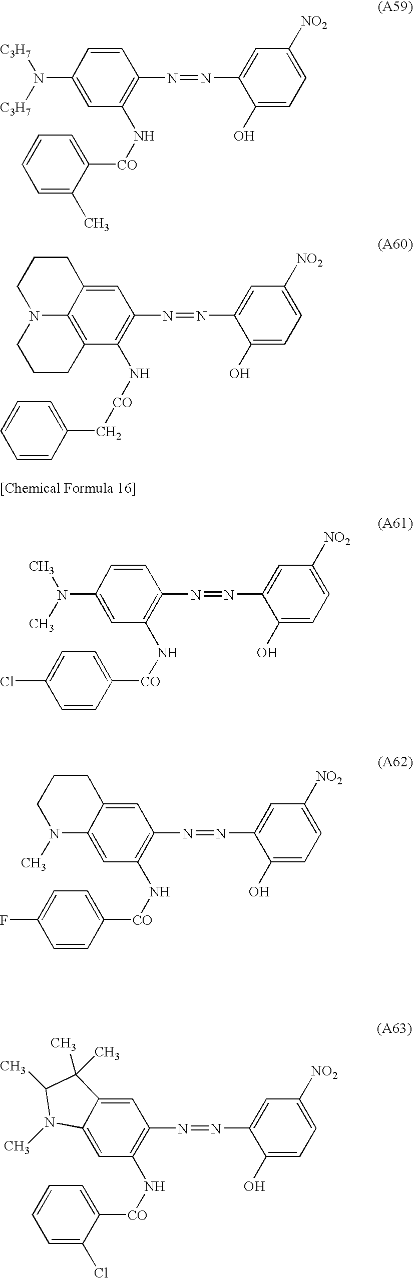

As metals (central metals) composing the chelate compound there are preferred transition metals such as Co, Mn, Cr; Ti, V, Ni, Cu, Zn, Mo, W, Ru, Fe, Pd, Pt and Al. Alternatively, V, Mo and W may be used as their oxide ions VO²⁺, VO³⁺, MoO²⁺, MoO³⁺ and WO³⁺. Particularly preferred among these are VO²⁺, VO³⁺, Co, Ni and Cu.

The chelate compound will normally have the azo compound as a bidentate or tridentate ligand forming coordination bonds with the metal. When the azo compound has a substituent with active hydrogen, the active hydrogens will generally dissociate to form a bidentate or tridentate ligand.

The chelate compound will sometimes be neutral overall, or will sometimes be an anion or cation. When the chelate compound is an anion, it will usually form a salt with its counter cation. As counter cations there may be mentioned metal cations such as Na⁺, Li⁺ and K⁺, and ammonium, tetraalkylammonium or the like. Alternatively, it may form a salt using the trimethinecyanine pigment cation as the counter cation, as mentioned above.

As specific preferred examples of chelate compounds there may be mentioned chelate compound Nos. C1 to C49 formed by coordination of the azo compound with the central metals in the combinations listed in Table 1, and any one of these or combination of two or more thereof may be used. In the chelate compounds listed in Table 1, two azo compounds are coordinated for each central metal element. Where two different azo compounds or central metals are shown in the table their molar ratio is 1:1, and “V═O” for the central metal indicates coordination of the azo compound with acetylacetone vanadium. These chelate compounds can be obtained by synthesis according to known methods (for example, see Furukawa, Anal. Chim. Acta., 140, p. 289, 1982).

TABLE 1

| | Azo | Central |
|---|---|---|
| No. | compound | metal |
| C1 | A1 | Co |
| C2 | A1 | V = O |
| C3 | A2 | Co |
| C4 | A2 | V = O |
| C5 | A3 | Co |
| C6 | A3 | V = O |
| C7 | A1 + A3 | Co |
| C8 | A1 + A2 | Co |
| C9 | A2 + A3 | Co |
| C10 | A1 | Co + |
| | | V = O |
| C11 | A2 | Co + |
| | | V = O |
| C12 | A3 | Co + |
| | | V = O |
| C13 | A4 | Cu |
| C14 | A4 | Ni |
| C15 | A4 | Co |
| C16 | A5 | Ni |
| C17 | A6 | Ni |
| C18 | A7 | Co |
| C19 | A7 | Ni |
| C20 | A7 | Cu |
| C21 | A8 | Co |
| C22 | A8 | Ni |
| C23 | A8 | Cu |
| C24 | A9 | Cu |
| C25 | A9 | Ni |
| C26 | A10 | Cu |
| C27 | A10 | Ni |
| C28 | A11 | Cu |
| C29 | A11 | Ni |
| C30 | A12 | Cu |
| C31 | A12 | Ni |
| C32 | A13 | Co |
| C33 | A14 | Co |
| C34 | A15 | Co |
| C35 | A16 | Co |
| C36 | A17 | Co |
| C37 | A18 | Co |
| C38 | A19 | Co |
| C39 | A20 | Co |
| C40 | A21 | Co |
| C41 | A22 | Co |
| C42 | A23 | Co |
| C43 | A24 | Co |
| C44 | A25 | Co |
| C45 | A26 | Co |
| C46 | A27 | Co |
| C47 | A28 | Co |
| C48 | A31 | Co |
| C49 | A32 | Co |

The content of the chelate compound in the optical recording material is preferably 10 to 70 mol % based on the total of the cation and chelate compound. The content is preferably 15 to 50 mol % and more preferably 20 to 30 mol %. A content of less than 10 mol % will tend to result in insufficient light stability, while a content of greater than 70 mol % will tend to increase jitter especially during high speed recording.

An optical recording material containing a trimethinecyanine pigment cation and chelate compound can be obtained by mixing the chelate compound with a salt comprising the trimethinecyanine pigment cation and its counter anion, or if the chelate compound is an anion, by forming a salt (salt-forming pigment) of the trimethinecyanine pigment cation and the chelate compound anion. The aforementioned mixture may also be used in combination with a salt-forming pigment.

The thickness of the first recording layer 20 and second recording layer 50 is preferably 50 to 300 nm. Outside of this range, the reflectance will be reduced and it will be difficult to achieve reproduction on the level of the DVD standard. The film thickness of the first recording layer 20 at the sections where it fills the groove 12 and the film thickness of the second recording layer 50 at the sections where it fills the groove 42 is preferably at least 100 nm and especially 130 to 300 nm from the standpoint of achieving a very high modulation factor.

The extinction coefficient (imaginary part k of the complex refractive index) of the first recording layer 20 and second recording layer 50 for the recording beam and reproducing beam is preferably 0 to 0.20. An extinction coefficient of greater than 0.20 will tend to result in insufficient reflectance. The refractive index (real part n of the complex refractive index) of the recording layer is preferably at least 1.8. A refractive index of less than 1.8 will tend to reduce the modulation factor of the signal. The upper limit for the refractive index is not particularly restricted but will normally be about 2.6 for convenience in synthesis of the organic pigment.

The first recording layer 20 and second recording layer 50 may be formed, for example, by a method of coating the base 10 or spacer layer 40 with a mixture comprising the optical recording material containing the pigment dissolved or dispersed in a solvent and removing the solvent from the coated film. As methods of coating the mixture there may be mentioned spin coating, gravure coating, spray coating, dip coating and the like, among which spin coating is preferred.

As solvents for the mixture there may be mentioned alcohol-based solvents (including alkoxy alcohol-based solvents such as ketoalcohol-based and ethyleneglycol monoalkyl ether-based solvents), aliphatic hydrocarbon-based solvents, ketone-based solvents, ester-based solvents, ether-based solvents, aromatic-based solvents, halogenated alkyl-based solvents and the like, among which alcohol-based solvents and aliphatic hydrocarbon-based solvents are preferred.

As alcohol-based solvents there are preferred alkoxy alcohol-based and ketoalcohol-based solvents. Alkoxyalcohol-based solvents preferably have 1-4 carbon atoms in the alkoxy portion and 1-5 and more preferably 2-5 carbon atoms in the alcohol portion, with a total of 3-7 carbon atoms. Specifically there may be mentioned ethyleneglycol monoalkyl ethers (cellosolves) such as ethyleneglycol monomethyl ether (methylcellosolve), ethyleneglycol monoethyl ether (also known as ethylcellosolve or ethoxyethanol), butylcellosolve, 2-isopropoxy-1-ethanol or the like, as well as 1-methoxy-2-propanol, 1-methoxy-2-butanol, 3-methoxy-1-butanol, 4-methoxy-1-butanol and 1-ethoxy-2-propanol. Diacetone alcohol may be mentioned as a ketoalcohol. Fluorinated alcohols such as 2,2,3,3-tetrafluoropropanol are also suitable for use.

As aliphatic hydrocarbon-based solvents there are preferred n-hexane, cyclohexane, methylcyclohexane, ethylcyclohexane, cyclooctane, dimethylcyclohexane, n-octane, iso-propylcyclohexane, t-butylcyclohexane and the like, among which ethylcyclohexane and dimethylcyclohexane are especially preferred.

Cyclohexanone may be mentioned as a ketone-based solvent.

Fluorinated alcohols such as 2,2,3,3-tetrafluoropropanol are particularly suitable for use in the present embodiment. Alkoxy alcohol-based solvents such as ethyleneglycol monoalkyl ether-based solvents are also preferred, among which ethyleneglycol monoethyl ether, 1-methoxy-2-propanol and 1-methoxy-2-butanol are especially preferred. The solvent may be a single type or a mixture of two or more different types. For example, a mixture of ethyleneglycol monoethyl ether and 1-methoxy-2-butanol may be suitably used. The mixture may also contain binders, dispersing agents, stabilizers and the like as appropriate in addition to the components mentioned above.

The semi-transparent reflective layer 30 is a layer having appropriate optical reflectance, as well as light transmittance of at least 40% for the recording and reproducing beams. The semi-transparent reflective layer 30 preferably has a certain degree of corrosion resistance. The semi-transparent reflective layer 30 also preferably has a barrier property, so that the material composing the spacer layer 40 does not seep into the first recording layer 20 and infiltrate the recording layer.

A highly reflective metal or alloy thin-film is preferably used as the semi-transparent reflective layer 30. For example, the material used for the semi-transparent reflective layer 30 may be a rare earth metal such as Au, Al, Ag, Cu, Ti, Cr, Ni, Pt, Ta, Pd, Mg, Se, Hf, V, Nb, Ru, W, Mn, Re, Fe, Co, Rh, Ir, Zn, Cd, Ga, In, Si, Ge, Te, Pb, Po, Sn or Bi, or an alloy containing any of these metals. Au, Al and Ag are preferred among the above as materials for the semi-transparent reflective layer 30 because of their high reflectance. Alloys containing at least 50% Ag, such as Ag—Bi alloy, are especially preferred. The concentration of Ag in the alloy is preferably 98-99.5 atom %.

In order to ensure high transmittance, the thickness of the semi-transparent reflective layer 30 is preferably no greater than 50 nm, more preferably no greater than 30 nm and even more preferably no greater than 20 nm. However, because a certain degree of thickness is necessary to prevent the first recording layer 20 from being affected by the spacer layer 40, it is preferably at least 3 nm and more preferably at least 5 nm.

The semi-transparent reflective layer 30 may be formed by, for example, sputtering, ion plating, chemical vapor deposition, vacuum vapor deposition or the like.

The spacer layer 40 is a transparent layer that separates the semi-transparent reflective layer 30 and second recording layer 50. A groove 42 for the second recording layer 50 is also formed on the second recording layer 50 side of the spacer layer 40, similar to the base 10. In order to apply a focus servo separately to the first recording layer 20 and second recording layer 50, the thickness of the spacer layer 40 is thickened to some degree to maintain distance between the recording layers. Specifically, the film thickness of the spacer layer 40 is preferably at least 5 [symbol] m and more preferably at least 10 [symbol] m. If the spacer layer 40 is too thick, time will be needed to match the focus servo to the two recording layers, while the moving distance of the objective lens will also be increased and more time will be necessary for curing, thus resulting in lower productivity, and therefore the spacer layer 40 thickness is preferably no greater than 100 [symbol] m.

The spacer layer 40 is formed of a resin such as, for example, a thermoplastic resin or thermosetting resin. The spacer layer 40 may be a single layer or it may have a multilayer structure. The spacer layer 40 may be formed, for example, by coating a semi-transparent reflective coat 30 with an uncured thermosetting resin or a coating solution obtained by dissolving it in a solvent, and then drying the coated film and exposing it to heat and light if necessary. The groove 42 may be formed by the 2P method at this time. The coating method used may be spin coating, casting, screen printing or the like.

The reflective layer 60 is provided to reflect the recording beam and reproducing beam. A metal or alloy thin-film may be used as the reflective layer 60. As metals and alloys there may be mentioned gold (Au), copper (Cu), aluminum (Al), silver (Ag), AgCu and the like. The thickness of the reflective layer 60 is preferably 10 to 300 nm. The reflective layer 60 may be formed by vapor deposition, sputtering or the like.

The adhesive layer 70 is a layer that bonds the dummy base 80 and reflective layer 60. The film thickness of the adhesive layer 70 in most cases is preferably at least 2 [symbol] m and more preferably at least 5 [symbol] m in order to ensure sufficient adhesive force while maintaining adequate productivity. The adhesive layer 70 is formed using a hot-melt adhesive, ultraviolet curing adhesive, heat curable adhesive, self-adhesive or pressure-sensitive double-sided tape.

The optical recording medium of the invention is not limited to the construction described above, of course. For example, a protective layer may be provided between the adhesive layer 70 and reflective layer 60 to prevent penetration of the reflective layer 60 by the material of the adhesive layer 70. Also, a publicly known inorganic or organic interlayer, adhesive layer or the like may be provided between the semi-transparent reflective layer 30 and first recording layer 20 or between the semi-transparent reflective layer 30 and spacer layer 40 for enhanced reflectance, improved recording characteristics and greater adhesiveness. The recording layer may be a single layer or three or more layers.

### EXAMPLES

The invention will now be explained in greater detail by examples and comparative examples. However, the present invention is not limited to the examples described below.

### Example 1

An optical recording material composed of a salt of the trimethinecyanine pigment of formula (T20) above (hereinafter referred to as “pigment T20”) and the chelate compound No. C5 in Table 1 (hereinafter referred to as “pigment C5”) was dissolved in 2,2,3,3-tetrafluoropropanol to a concentration of 1.0 wt % to prepare a mixture. The mixture was coated onto a polycarbonate resin base having a pregroove (depth: 0.16 [symbol] m, width: 0.30 [symbol] m, groove pitch: 0.74 [symbol] m) formed therein, and dried to form a first recording layer (thickness: 130 nm, hereinafter referred to as “L₀”). Next, a semi-transparent reflective layer (thickness: 15 nm) made of Ag—Bi alloy was formed on L₀ by sputtering, and a spacer layer having a groove formed on the surface using a stamper made of a polyolefin transparent resin (depth: 0.17 [symbol] m, width: 0.30 [symbol] m, groove pitch: 0.74 [symbol] m) was formed on the semi-transparent reflective layer using an ordinary adhesive. Next, the same optical recording material as L₀ was used to form a second recording layer (thickness: 130 nm, hereinafter referred to as “L₁”) on the groove-formed spacer layer, and a reflective layer made of Ag (thickness: 85 nm) was formed thereover by sputtering. A transparent protective layer (thickness: 5 [symbol] m) made of an ultraviolet curing acrylic resin was then formed on the reflective layer to obtain an optical recording disk possessing two recording layers.

The obtained optical recording disk was used for recording of a signal at a linear speed of 3.84 m/s (corresponding to 1×) using laser light with a wavelength of 655 nm, and the signal was reproduced at a linear speed of 3.84 in/s using laser light with a wavelength of 650 nm, during which time the jitter was measured. The lens aperture NA was 0.60. For durability testing, the obtained optical recording disk was allowed to stand for 100 hours in an environment of 80° C., 80% humidity and then again measured for jitter. The results are summarized in Table 2.

### Example 2

An optical recording disk was fabricated and evaluated in the same manner as Example 1, except that L₀ and L₁ were formed using an optical recording material obtained by mixing a salt of pigment T20 and pigment C5 and a PF₆⁻ salt of pigment T20 in a weight ratio of 60:40.

### Example 3

An optical recording disk was fabricated and evaluated in the same manner as Example 1, except that L₀ was formed using an optical recording material obtained by mixing a salt of pigment T20 and pigment C5 and a PF₆⁻ salt of the trimethinecyanine pigment of formula (T55) above (hereinafter referred to as “pigment T55”) in a weight ratio of 60:40, and L₁ was formed using an optical recording material obtained by mixing a salt of pigment T20 and pigment C5 with a PF₆⁻ salt of pigment T55 in a weight ratio of 70:30.

### Example 4

An optical recording disk was fabricated and evaluated in the same manner as Example 1, except that L₀ was formed using an optical recording material obtained by mixing a salt of pigment T20 and pigment C5 and a PF₆⁻ salt of pigment T20 in a weight ratio of 60:40, and L₁ was formed using an optical recording material obtained by mixing a salt of pigment T20 and pigment C5 with a PF₆⁻ salt of pigment T55 in a weight ratio of 65:35.

### Example 5

An optical recording disk was fabricated and evaluated in the same manner as Example 1, except that L₀ was formed using an optical recording material composed of a salt of pigment T20 and pigment C5, and L₁ was formed using an optical recording material obtained by mixing a salt of pigment T55 and pigment C5 with a PF₆⁻ salt of pigment T20 in a weight ratio of 50:50.

### Comparative Example 1

An optical recording disk was fabricated and evaluated in the same manner as Example 1, except that L₀ and L₁ were both formed using an optical recording material composed of a salt of a trimethinecyanine pigment represented by the following formula (T0) (hereinafter referred to as “pigment T0”) and pigment C5.

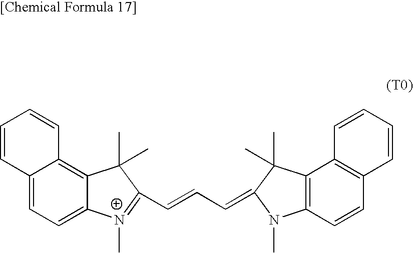

### Comparative Example 2

An optical recording disk was fabricated and evaluated in the same manner as Example 1, except that L₀ and L₁ were both formed using an optical recording material obtained by mixing a salt of pigment T0 and pigment C5 with a PF₆⁻ salt of pigment T0 in a weight ratio of 60:40.

TABLE 2

| | | | | | Jitter | | | |
|---|---|---|---|---|---|---|---|---|
| | | | | | | | After | |
| | L₀ | | L₁ | | | | durability | |
| | | Weight | | Weight | Initial | | test | |
| | Pigment | ratio | Pigment | ratio | L₀ | L₁ | L₀ | L₁ |
| Example | T20•C5 | 100 | T20•C5 | 100 | 6 | 6.2 | 6 | 7.3 |
| 1 | | | | | | | | |
| Example | T20•C5 | 60 | T20•C5 | 60 | 6.2 | 6 | 6.2 | 7.5 |
| 2 | T20•PF₆⁻ | 40 | T20•PF₆⁻ | 40 | | | | |
| Example | T20•C5 | 60 | T20•C5 | 70 | 6.2 | 6.2 | 6.3 | 7.8 |
| 3 | T55•PF₆⁻ | 40 | T55•PF₆⁻ | 30 | | | | |
| Example | T20•C5 | 60 | T20•C5 | 65 | 5.9 | 6.1 | 6 | 7.5 |
| 4 | T20•PF₆⁻ | 40 | T55•PF₆⁻ | 35 | | | | |
| Example | T55•C5 | 100 | T55•C5 | 50 | 6.2 | 6.5 | 6.2 | 7.4 |
| 5 | | | T20•PF₆⁻ | 50 | | | | |
| Comp. | T0•C5 | 100 | T0•C5 | 100 | 9 | 9 | 11 | 15.3 |
| Ex. 1 | | | | | | | | |
| Comp. | T0•C5 | 60 | T0•C5 | 60 | 8.5 | 8.9 | 12 | 16 |
| Ex. 2 | T0•PF₆⁻ | 40 | T0•PF₆⁻ | 40 | | | | |

As shown in Table 2, the optical recording disk of the examples using the trimethinecyanine pigment with an allyl group exhibited excellent jitter characteristics. Excellent jitter characteristics were also maintained after durability testing under high moist heat conditions, thus confirming that the preservation stability was also excellent. In contrast, the optical recording disks of the comparative examples using the trimethinecyanine pigment without allyl groups had unsatisfactory jitter and notably reduced jitter characteristics after durability testing. It was thus confirmed that the invention provides an optical recording medium that has satisfactory sensitivity while exhibiting adequate characteristics in terms of jitter and preservation stability.

1-4. (canceled)

5. An optical recording material for use in an optical recording medium that allows recording of information by light exposure, the optical recording material comprising:
a cation represented by the following general formula (1); and
a chelate compound of an azo compound represented by the following general formula (2) and a metal. 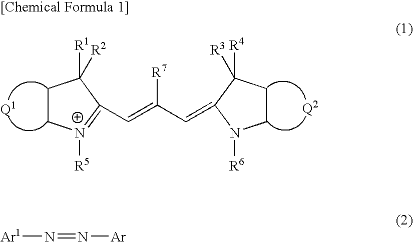
[In formula (1),
R1 and R2 each independently represent a monovalent group represented by Chemical Formula (10) below, a C1-4 alkyl group, an optionally substituted benzyl group, or a group linking together to form a 3- to 6-membered ring,
R3 and R4 each independently represent a monovalent group represented by Chemical Formula (10) below, a C1-4 alkyl group, an optionally substituted benzyl group, or a group linking together to form a 3- to 6-membered ring,
R5 and R6 each independently represent an optionally substituted alkyl group or an optionally substituted aryl group,
R7 represents a hydrogen atom, a halogen atom, a cyano group, an optionally substituted alkyl group or an optionally substituted aryl group,
Q1 and Q2 each independently form an optionally substituted benzene ring or an optionally substituted naphthalene ring, and
at least one from among R1, R2, R3 and R4 is a monovalent group represented by Chemical Formula (10) below:
[Chemical Formula 2]
[CH₂═CH—CH₂ (10)
In formula (2),
Ar1 and Ar2 each independently represent an optionally substituted aryl group, and at least one of Ar1 and Ar2 is an aryl group with a substituent capable of coordinating with a metal atom or an aryl group composed of an optionally substituted nitrogen-containing heteroaromatic ring with a nitrogen atom capable of coordinating with a metal atom.]

6. An optical recording material according to claim 5,
obtainable by mixing a salt of the cation and its counter anion with the chelate compound.

7. An optical recording medium that allows recording of information by light exposure, the optical recording medium being provided with a recording layer comprising:
a cation represented by the following general formula (1); and
a chelate compound of an azo compound represented by the following general formula (2) and a metal. 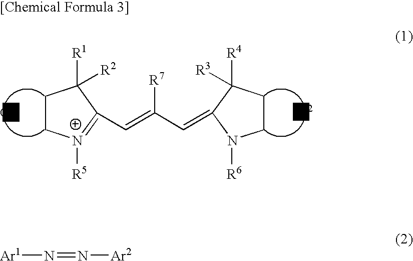
[In formula (1),
R1 and R2 each independently represent a monovalent group represented by Chemical Formula (10) below, a C1-4 alkyl group, an optionally substituted benzyl group, or a group linking together to form a 3- to 6-membered ring,
R3 and R4 each independently represent a monovalent group represented by Chemical Formula (10) below, a C1-4 alkyl group, an optionally substituted benzyl group, or a group linking together to form a 3- to 6-membered ring,
R5 and R6 each independently represent an optionally substituted alkyl group or an optionally substituted aryl group,
R7 represents a hydrogen atom, a halogen atom, a cyano group, an optionally substituted alkyl group or an optionally substituted aryl group,
Q1 and Q2 each independently form an optionally substituted benzene ring or an optionally substituted naphthalene ring, and
at least one from among R1, R2, R3 and R4 is a monovalent group represented by Chemical Formula (10) below:
[Chemical Formula 2]
[CH₂═CH—CH₂ (10)
In formula (2),
Ar1 and Ar2 each independently represent an optionally substituted aryl group, and at least one of Ar1 and Ar2 is an aryl group with a substituent capable of coordinating with a metal atom or an aryl group composed of an optionally substituted nitrogen-containing heteroaromatic ring with a nitrogen atom capable of coordinating with a metal atom.]

8. An optical recording medium according to claim 7,
wherein the recording layer comprising a mixture obtainable by mixing a salt of said cation and its counter anion with said chelate compound.
